(19) United States
(12) Patent Application Publication
(10) Pub. No.: US 2008/0311598 A1
(43) Pub. Date: Dec. 18, 2008

(54) Magnetic Biosensor For Determination of Enzymic Activity

(75) Inventors: Erik Robbert Vossenaar, Eindhoven (NL); Menno Willem Jose Prins, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/094,822

(22) PCT Filed: Nov. 21, 2006
(86) PCT No.: PCT/IB2006/054346

§ 371 Date: May 23, 2008

(30) Foreign Application Priority Data
Nov. 25, 2005 (EP) 05111301.7

(51) Int. Cl. G01N 33/543; C12M 1/34

(52) U.S. Cl. 435/7.72; 435/287.1

(57) ABSTRACT

Magnetic sensors are very suitable for use in determination of enzymatic activity. In a preferred embodiment the invention relates to a method for determining activity of an enzyme in modification of substrate (2) to product (3), comprising binding substrate or a binding composition that may bind e.g. substrate or product, to the sensor surface. This enables easy detection of magnetic label that is linked to substrate, product or the binding composition.

### FIELD OF THE INVENTION

The invention relates to the field of detection or diagnostics, especially biomolecular diagnostics for both in vivo and in vitro application. More particularly the invention relates to a magnetic sensing device using magnetic particles to carry out an enzymatic assay.

### BACKGROUND TO THE INVENTION

In the field of diagnostics e.g. medical diagnostics, sensors are used to carry out all kinds of assays. In these assays concentration of a specific target material may be determined. The assays are also used to monitor enzymatic conversions.

Examples of well-known targets are DNA, RNA, peptides, proteins, drugs, pathogens, hormones, sugar, and cellular material. Labels are generally used in the assay to enable determination of the concentration of the target. Currently used labels are optical labels and fluorescent labels.

In the detection of certain diseases it is desirable to determine enzyme activity in a complex biological sample. Not all assay set ups are suitable for this and problems may arise with respect to sensitivity of the assay, non-specific binding, too slow binding which leads to an inaccurate outcome of the measured enzyme activity or complicated pre-purification steps which are not user-friendly.

Furthermore many known assays for determining enzyme activity use an artificial substrate that provides a colour effect in reaction with the enzyme. This colour effect is used to determine enzyme activity. It may be difficult to find such artificial substrates for a given enzyme and therefore it is desired that an assay is provided which does not solely rely on the availability of such artificial substrate.

It is an object of the invention to provide a sensing device that overcomes one or more of these drawbacks.

### SUMMARY OF THE INVENTION

We have surprisingly found that magnetic sensors are very suitable for use in enzymatic assays.

Therefore in one aspect the invention relates to a method for determining enzyme activity using a magnetic sensor.

In another aspect the invention relates to a device suitable for use in this method.

These and other aspects and features of the invention will become apparent from the following detailed description, taken in conjunction with the accompanying drawings, which illustrate, by way of example, the invention. The references quoted refer to the drawings.

### DESCRIPTION OF THE FIGURES

FIG. 1 illustrates an embodiment where enzyme (1) modifies substrate (2) to form product (3).

FIG. 2 illustrates an embodiment where enzyme (1) degrades substrate (2) to form product (3).

FIG. 3 illustrates an embodiment where the binding composition (4) is coupled to the sensor surface (6).

FIG. 4 illustrates an embodiment where conversion of substrate (2) to product (3) leads to cleavage of the substrate which then no longer binds to immobilized binding composition (4).

FIG. 5 illustrates the embodiment where the substrate is immobilized on a solid surface and the binding composition (4) is immobilized on the sensor surface.

FIG. 6 illustrates the embodiment where a magnetically labelled binding composition (9) binds the enzyme.

FIG. 7-10 illustrates a specific embodiment of the capture mechanism where the enzyme is captured by a magnetically labelled binding composition (9) and a magnetic field is applied to attract the captured enzyme to a surface that contains immobilized thereon a substrate.

### DETAILED DESCRIPTION

Where an indefinite or definite article is used when referring to a singular noun, e.g. “a”, “an”, “the”, this includes a plural of that noun unless something else is specifically stated.

It is to be noticed that the term “comprising” used in the present description and claims, should not be interpreted as restricted to the means listed thereafter; it does not exclude other elements or steps.

The current invention deals with use of a magnetic sensor for analysing enzyme activity. The method is especially suitable for use in analysing activity of enzymes that are present in complex biological samples. An advantage of the use of the invention is that it obviates the need for extensive pre-purification methods of the sample.

A further advantageous feature of the magnetic sensor for use in determination of enzyme activity is that kinetics of the enzymatic reaction can be determined directly, during the reaction. Therefore the current use and method are also suitable for enzyme-substrate affinity determination.

In the context of the invention, a magnetic sensor is defined as a sensor device, which uses magnetic labels to identify the presence of a certain composition.

Zhao et al (Angew Chem Intl Ed 2003, 42, No 12, pages 1375-1378) disclose the use of magnetic particles in an enzyme assay. Detection is based on the formation of magnetic particle clusters which are imaged with MR imaging using a 1.5 T superconducting magnet. Such MR imaging equipment is not encompassed in the definition of the magnetic sensor device according to the current invention. The devices according to the invention detect the presence of magnetic labels that are bound to a sensor surface.

We will discuss the present invention for a range of specific embodiments thereof.

According to one aspect, the invention relates to a method of analysing enzyme activity using a magnetic sensor device wherein the activity of enzyme (1) to modify a substrate (2) to form product (3) is determined by using a binding composition (4) and a magnetic label (5), the method comprising the steps of

(a) immobilizing the substrate (2) or the binding composition (4) on a sensor surface

(b) contacting the substrate (2) and the enzyme (1),

(c) providing a magnetic label to the binding composition or the substrate

(d) contacting the binding composition with substrate (2) or product (3) to allow direct or indirect linking of the binding composition (4) to the substrate (2) or the product (3),

(e) detecting the magnetic label with a magnetic sensor.

These steps may be carried out in any suitable order. In a preferred embodiment of this method, the substrate or binding composition is immobilized (step a) before contacting the substrate with the enzyme.

Preferably, in a separate preparatory step, a magnetic label is provided to the binding composition or the substrate.

In a preferred sequence of events, step (d), (e) are carried out after step (a), (b).

In a step of the invention, a magnetic label is provided to a binding composition and/or the substrate. It is preferred that the bond between the label and the binding composition or substrate is covalent, but other types of bonds such as hydrogen bonding are also possible.

The magnetic label is also referred to as magnetic beads, or particles. The magnetic labels are preferably spherical in shape but this is not a requirement for all embodiments. Other suitable shapes are e.g. cylinders, rods, cubes, and ovals.

By the term “magnetic label” it is understood that the labels include any suitable form of one or more magnetic particles e.g. magnetic, diamagnetic, paramagnetic, superparamagnetic, ferromagnetic that is any form of magnetism which generates a magnetic dipole in a magnetic field, either permanently or temporarily. Examples of suitable magnetic label material are: Fe₃O₄ beads.

The size of the magnetic label is not critical in most embodiments but for many biosensor applications it is highly preferred that the labels are of a small size. Preferred magnetic labels have size (generally expressed as longest diameter) of from 1 to 3000 nm, more preferred from 5 to 500 nm, even more preferred from 10 to 300 nm, most preferred from 30 to 250 nm.

Detection of a magnetic label is generally done by application of an electric, or magnetic, or electromagnetic field.

The current invention relates to determination of enzymatic activity. Suitable enzymes of which activity can be determined with this method are for example selected from the group comprising the classes of caspases, proteases, kinases. The method and use are specifically suitable for determining activity of enzymes that require a cofactor that is present in a biological sample containing the enzyme but which may not survive extensive pre-purification procedures that are common for other detection methods than magnetic detection.

The enzymes may be present in samples (7) that have been specifically prepared for this purpose or may be part of a “raw material” that is to be analysed to determine its enzymatic activity with respect to a specific substrate. One of the advantages of the claimed method is that a pre-purification of “raw material” such as body fluids including blood and urine is not needed to obtain reliable data.

The activity of an enzyme is generally expressed in units. Units are generally defined as the amount of enzyme that is required to convert a certain amount (moles or grams) of substrate in a certain time frame (minutes or hours). Specific activity may also be expressed as units per volume of sample.

In the context of the invention “degrading enzymes” are defined as enzymes that facilitate the cleavage (e.g. endopeptidases or endonucleases) or breakdown (e.g. exopeptidases or exonucleases) of a substrate (2) into a product (3), which is a degradation product. Generally cleavage results in the release of at least two product parts.

In the method, use and device of the invention, binding compositions are used.

A “binding composition” is a composition that binds to another component with a measurable strength that is larger than the strength of non-specific binding.

Binding compositions can be peptides or proteins with binding affinity for a substrate or product. Examples of binding compositions are antibodies or functional fragments and derivatives thereof such as Fab fragments, single chain Fv, VHH, heavy chain antibodies. Antibodies can be raised to non-proteinaceous compounds as well as to proteins or peptides. A binding composition preferably binds with high specificity, with a high affinity and the bond with the substrate or product is preferably such that it can withstand assay conditions. Other examples of binding compositions are receptor proteins, ligands, aptamers, oligonucleotides, or functional fragments or derivatives of any of these.

In a preferred embodiment of the invention, the substrate is a protein or peptide because these may easily be converted into product by an enzymatic reaction. Alternative substrates are other biological and chemical substances such as nucleic acids, lipids, carbohydrates and chelators.

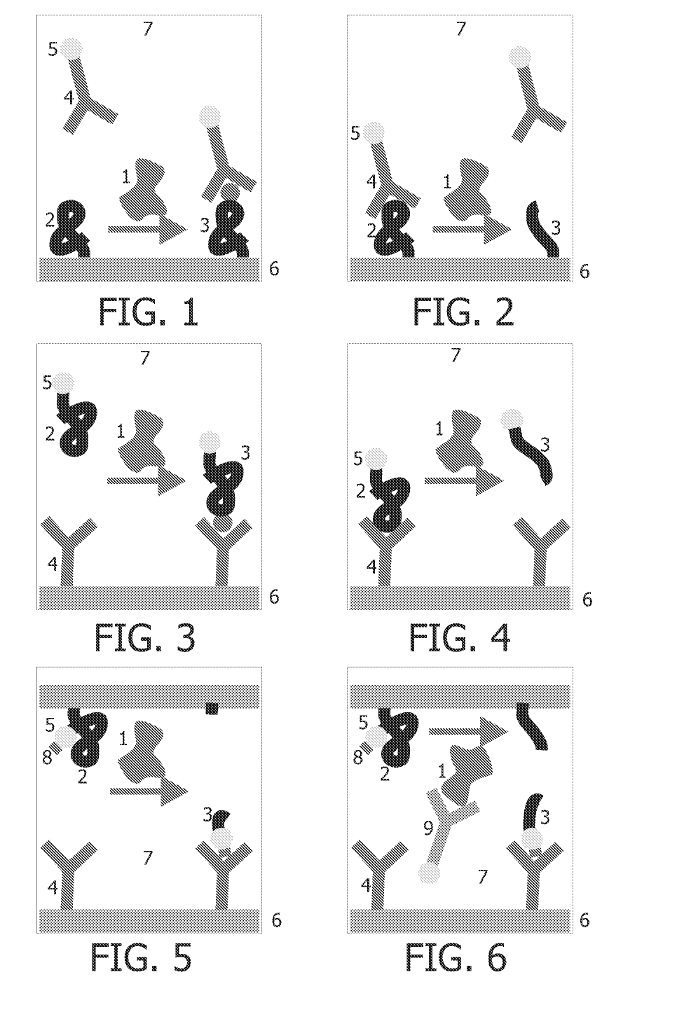

FIG. 1 illustrates a first preferred embodiment of the invention. In this embodiment, the activity of enzyme (1) to modify substrate (2) to form product (3) is determined. In this specific embodiment, the substrate (2) is immobilized on a sensor surface (6). A sample (7) comprising substrate-modifying enzyme (1) is contacted with the sensor surface. Depending on the reaction conditions, but assuming these are right, the substrate (2) will be converted to some extent to product (3) unless the enzyme is fully inactive. In this embodiment, product (3) remains attached to the sensor surface. Binding composition (4) is labeled with a magnetic label. Binding composition (4) binds specifically to product (3) which product remains immobilized on the sensor surface. A magnetic sensor, e.g. a magneto resistive sensor embedded in the sensor surface, may detect the binding of the magnetic particle to the label. The amount of product (3) and hence the activity of the enzyme is proportional to the amount of label that is detected.

The sample (7) is preferably a fluid sample. An aqueous composition is highly suitable for use in this method.

In an alternative embodiment, binding composition (4) does not directly bind to product (3) but binds to a primary binding composition (8) which primary binding composition (8) directly binds to the product (3). In that case detection can occur after primary binding moiety (8) is exposed and coupled to a magnetic entity linked to another binding composition (4). Examples of this embodiment include the method where the primary binding moiety interacts with a binding moiety that carries a magnetic label via a ligand/binder interaction such as biotin-streptavidin, hapten/anti-hapten, or digoxygenin/anti-digoxygenin interaction.

In FIG. 2 another embodiment is illustrated. In this embodiment, substrate (2) is degraded by an enzyme (1). In this embodiment it may be difficult to measure the product that results from the degradation after contact with the enzyme. Therefore in this embodiment, the binding composition binds directly or indirectly to the substrate. In this situation, the substrate is a so-called unmodified substrate. The amount of binding composition that is found after incubation with an enzyme-containing sample (7) is inversely correlated with the enzymatic activity that is present in the sample. In this embodiment, it is preferred that the magnetic label is linked to the binding composition. In an even more preferred embodiment, the binding composition is directly binding to the substrate.

In an alternative embodiment, the invention relates to a method of analysing enzyme activity using a magnetic sensor device wherein the activity of enzyme (1) to modify a substrate (2) to form product (3) is determined, comprising the steps of

(a) immobilizing a binding composition capable of binding to substrate (2) or product (3)

(b) providing substrate (2) with a magnetic label

(c) contacting the substrate (2) and the enzyme

(d) contacting the binding composition with substrate (2) or product (3) to allow direct or indirect linking of the binding composition (4) the substrate (2) or the product (3)

(e) detecting the magnetic label with a sensor, preferably a magnetic sensor.

One aspect of this embodiment is illustrated in FIG. 3. In this embodiment, the substrate is coupled to a magnetic label directly or indirectly and the binding composition (4) is coupled to the sensor surface (6). The binding composition specifically binds product (3), which still includes the magnetic label. The binding composition (4) does not or to a much lesser extent bind to the substrate (2). This method is especially suited for small substrates. Without wishing to be bound by any theory it is believed that this method diminishes steric hindrance in enzyme-substrate interaction from the sensor surface.

Another embodiment is described in FIG. 4. In this embodiment, a binding composition (4) capable of binding substrate (2) carrying magnetic label (5) is immobilized on the sensor surface. The substrate is contacted with a sample comprising enzyme (1). Conversion of the substrate (2) into product (3) leads to cleavage of the substrate, which no longer binds to the binding composition. The amount of magnetic label that is detected after the reaction is inversely related to the enzymatic activity that is present in the sample. This embodiment is especially suitable for use in the analysis of activity of degrading enzymes.

Therefore in a preferred embodiment the invention relates to a method as identified above, wherein

(a) the enzyme is a degrading enzyme and

(b) the binding composition binds substrate (2)

(c) the enzymatic degrading reaction leads to the formation of a composition (3b) which comprises the magnetic label and which does not bind to the binding composition.

In a further embodiment, to provide improved control over reaction conditions and concentrations of substrate, product and enzyme, both the substrate and the binding composition (4) are immobilized on a surface. Preferably they are each immobilized on a different surface being in each other's vicinity, even more preferred the surfaces are essentially parallel to each other and facing each other.

This embodiment is characterised by the following features:

a) the substrate (2) and the binding composition (4) are immobilised on a surface and;

b) the substrate comprises a magnetic label and

c) the enzyme activity results in a degrading of the substrate such that product (3) comprises the magnetic label

d) product (3) binds to the binding composition (4) which is preferably positioned on a surface facing the sensor surface to which the substrate is immobilized.

In this embodiment it is preferred that the substrate is immobilized on a solid surface and the binding composition (4) is immobilized on the sensor surface to enable detection of magnetic label that is bound to the product (3) which binds to the binding composition (4). This embodiment is illustrated in FIG. 5.

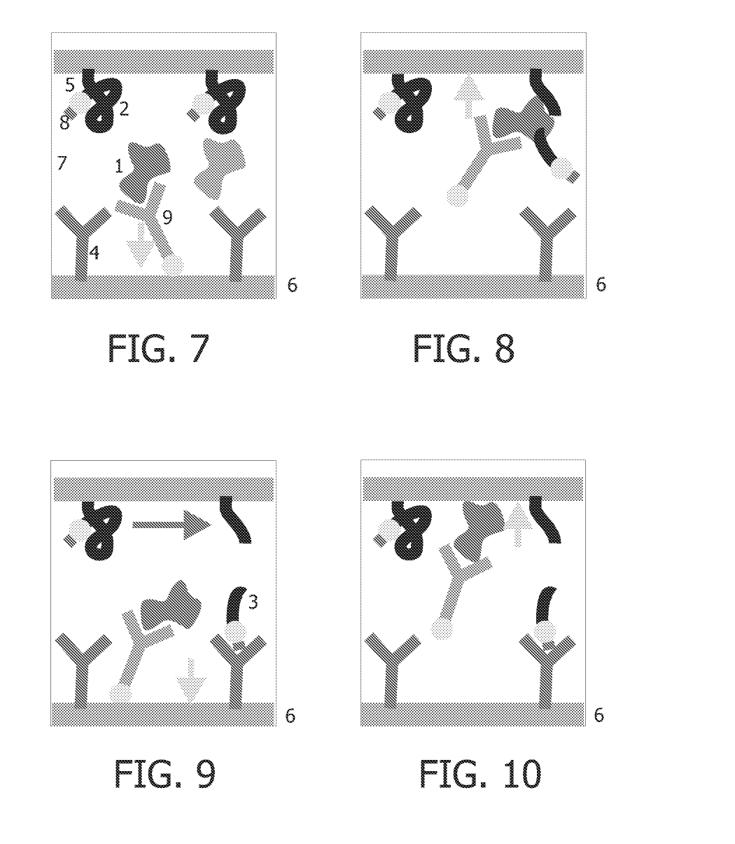

Optionally in this embodiment, a second binding component (9) provided with a magnetic label binds the enzyme. This enables one to steer the reaction by application of magnetic fields to capture the enzyme or bring it into a certain space of the sensor. This is illustrated in FIG. 6 and more specifically in FIGS. 7-10.

In FIG. 6, a binding composition (9) binding enzyme (1), is coupled to a magnetic particle, and captures the enzyme. This has the advantage that an integrated separation can be done in the sensor system, which allows distinguishing between activities of various subtypes of enzyme with the same enzyme activity provided specific binding compositions are available for each subtype.

FIGS. 7-10 illustrate a specific embodiment of the capture mechanism. This method allows for discrimination of the activity of different enzymes with the same enzymatic activity (e.g. salivary amylase and pancreatic amylase). In FIG. 7, a magnetically labelled binding composition captures an enzyme of interest. A magnetic field is then applied to attract the captured enzyme to the surface that comprises immobilized substrate. Enzymes that are not captured are not attracted to this surface and are washed out in this step. By actuation the captured enzyme can be brought towards the surface containing the immobilized substrate (FIG. 8). The substrate is then enzymatically converted into a product (3) in a degradation reaction. In this degradation a haptenized magnetic label is released. Again by actuation, the label is brought to the sensor surface, which comprises immobilized binding composition that binds to the hapten that is present on product (3), which is released in the enzymatic reaction. This is illustrated in FIG. 9. The label thus binds to the immobilized binding composition. In an optional further step, non haptenized magnetic labels can be removed by changing the direction of the magnetic field such that only strongly bound magnetic labels linked to immobilized binding composition (4) remain in place. This is illustrated in FIG. 10. The amount of bound haptenized magnetic label is used to calculate the enzymatic activity present in the sample.

According to another embodiment, in the method according to the invention, enzyme activity is determined in the following way. A substrate (2) is provided with a magnetic label. The substrate is also linked to a first element of a primary binding composition (8). The primary binding composition (8) binds preferentially to a binding composition (4) which is attached to a sensor surface. Active enzyme cleaves the substrate such that a separate primary binding composition (8) and a separate magnetic label, result. The rate of binding of the magnetic label to the sensor surface will then reduce due to two mechanisms: a) primary binding composition (8) is no longer coupled to the magnetic label and (b) cleaved units of primary binding composition (8) bind to the binding composition (4). As a result the binding rate of magnetic labels to the binding moieties on the sensor surface is a measure of the enzyme activity. A high binding rate indicates a low enzyme activity. Optionally the methods as described above comprise further steps such as described below.

Optionally a wash out step is included. This wash out step may be used to remove non-specifically bound magnetic label, enzyme, and other components.

In another aspect the invention relates to a magnetic sensor device suitable for determination of enzymatic activity.

This device preferably comprises

(i) At least one electric or magnetic field generating means (10) for applying an electric or magnetic field to a sample fluid containing magnetic label

(ii) At least one magnetic sensor element (11)

(iii) At least one sensor surface

(iv) At least one inlet for fluid compositions

(v) At least one outlet for fluid compositions.

Preferably the device further comprises a reaction chamber for carrying out the enzymatic reaction.

In general a magnetic sensing device will be sensitive to magnetic labels that are specifically attached to the sensor surface and to some extent as well to labels that are not specifically attached but are in close vicinity of the sensor surface. To make the device highly specific for magnetic label that is bound to the sensor surface, the device preferably comprises a sensor surface comprising immobilized thereon a substrate or a binding composition.

The substrate and binding composition may also be provided separately in dry or wet form.

In a preferred embodiment, especially for carrying out the method illustrated in FIG. 5-10, the device comprises a sensor surface comprising immobilized binding composition binding product (3), preferably an antibody binding product (3), and a solid surface comprising immobilized substrate (2). It is preferred that the solid surface comprising immobilized substrate (2) is in close vicinity to the sensor surface.

Optionally the device comprises means to control temperature and or means for cleaning.

The invention further relates to a chip comprises at least one of the devices as described above, more preferred a multitude of devices as described above.

The magnetic sensor element may be any suitable element but is preferably selected from an AMR (anisotropic magneto-resistance), GMR (giant magneto-resistanca) or a TMR (tunneling magneto-resistance) sensor element. Sensor elements based on other principles such as Hall sensor elements or SQUIDS are also possible for application in the claimed device.

The reaction chamber may a separate entity in the device or may be an in line reaction place. The reaction chamber is the place in the device where the contact between sample (7) and enzyme (1) takes place.

The device or chip preferably comprises a base on which the described components are placed. This base can be made of any suitable material e.g. glass, plastic or a combination of these.

In a further aspect the invention relates to a kit of parts comprising the device as specified above in combination with at least one binding composition and a substrate. Such device is suitable for enzyme activity analysis of an enzyme that may convert the substrate. The suitable substrate and binding composition may be provided as separate parts of the kit of parts or may be integrated in the device.

Preferably at least one of the substrate and the binding composition is immobilized on a surface (more preferred the sensor surface) of the device.

Optionally this kit of parts further comprises other components such as reaction medium, cofactors.

The device, methods and systems of this invention are suited for sensor multiplexing (i.e. the parallel use of different sensors and sensor surfaces), label multiplexing (i.e. the parallel use of different types of labels) and chamber multiplexing (i.e. the parallel use of different reaction chambers).

The device, methods and systems described in the present invention can be used as rapid, robust, and easy to use point-of-care biosensors for small sample volumes. The device preferably comprises a reaction chamber which may be a disposable item to be used with a compact reader, containing the one or more magnetic field generating means and one or more detection means. Also, the device, methods and systems of the present invention can be used in automated high-throughput testing. In this case, the reaction chamber is e.g. a well plate or cuvette, fitting into an automated instrument.

1. Method of analysing enzyme activity using a magnetic sensor device wherein the activity of enzyme to modify a substrate to form product is determined by using a binding composition and a magnetic label, the method comprising the steps of
(a) immobilizing the substrate or the binding composition on a sensor surface
(b) contacting the substrate and the enzyme
(c) providing a magnetic label to the binding composition or the substrate
(d) contacting the binding composition with substrate or product to allow direct or indirect linking of the binding composition to the substrate or the product,
(e) detecting the magnetic label with a magnetic sensor.

2. Method according to claim 1, wherein the activity of enzyme to modify substrate to product is determined and wherein the substrate is immobilized on a sensor surface.

3. Method according to claim 2 wherein the binding composition directly binds to the product.

4. Method according to claim 2 wherein the binding composition binds to a primary binding composition which primary binding composition directly binds to the product.

5. Method according to claim 1 wherein the activity of enzyme to degrade substrate is determined, and wherein the binding composition binds directly or indirectly to the substrate.

6. Method according to claim 5 wherein the binding composition binds to a primary binding composition which primary binding composition directly binds to the substrate.

7. Method of analysing enzyme activity using a magnetic sensor device wherein the activity of enzyme to modify a substrate to form product is determined, comprising the steps of
(a) immobilizing a binding composition capable of binding to substrate or product
(b) providing substrate with a magnetic label
(c) contacting the substrate and the enzyme
(d) contacting the binding composition with substrate or product to allow direct or indirect linking of the binding composition to the substrate or the product respectively,
(e) detecting the magnetic label with a magnetic sensor.

8. Method according to claim 7 wherein
(a) the enzyme is a degrading enzyme and
(b) the binding composition binds substrate
(c) the enzymatic degrading reaction leads to the formation of a composition which comprises the magnetic label and which does not bind to the binding composition.

9. A method according to claim 1 for determining the activity of a degrading enzyme wherein
a) the substrate and the binding composition are immobilised on a surface and;
b) the substrate comprises a magnetic label and
c) the enzyme activity results in a degrading of the substrate such that product comprises the label
d) product binds to the binding composition.

10. Method according to claim 9, wherein a second binding component provided with a magnetic label binds the enzyme and magnetic fields can be applied to capture the enzyme or bring it into a certain space of the sensor.

11. A method according to claim 10 wherein the substrate is immobilized on a solid surface and the binding composition is immobilized on the sensor surface of the magnetic biosensor.

12. Device for analysing enzymatic activity of enzyme in the modification of substrate to product, comprising
i. At least one electric or magnetic field generating means for applying an electric or magnetic field to a sample fluid containing magnetic label
ii. At least one magnetic sensor element
iii. At least one sensor surface
iv. At least one inlet for fluid compositions
v. At least one outlet for fluid compositions.

13. Device according to claim 12 wherein a substrate and/or a binding composition are immobilized on the sensor surface (iii).

14. Device according to claim 13 which comprises a sensor surface comprising immobilized binding composition binding product, preferably an antibody binding to the product, and a solid surface comprising immobilized thereon, the substrate.

15. Chip comprising at least one of the device according to claim 12, more preferred a multitude of the device according to claim 12.

16. A kit of parts for enzyme analysis, comprising a device according to claim 12 in combination with a suitable enzyme substrate and/or a binding composition.
